(19) United States
(12) Patent Application Publication
(10) Pub. No.: US 2022/0146497 A1
(43) Pub. Date: May 12, 2022

(54) Method of Modulating a Fibrotic Condition

(71) Applicants: The Procter & Gamble Company, Cincinnati, OH (US); Agency For Science, Technology And Research, Singapore (SG)

(72) Inventors: Robert Joseph ISFORT, Fairfield, OH (US); Frederic BARD, Singapore (SG); Hui Hui WONG, Singapore (SG); Sze Hwee SEET, Singapore (SG)

(21) Appl. No.: 17/582,418

(22) Filed: Jan. 24, 2022

Related U.S. Application Data
(63) Continuation of application No. PCT/US2020/070440, filed on Aug. 21, 2020.
(60) Provisional application No. 62/890,641, filed on Aug. 23, 2019.

(51) Int. Cl. G01N 33/50

(52) U.S. Cl. CPC G01N 33/5023; G01N 33/5044

(57) ABSTRACT

A method of identifying fibrotic agents capable of modulating a fibrotic process or condition is provided, along with compositions containing such compounds. The method involves contacting a plurality of fibroblasts with a test compound and determining whether the compound modulates expression of a gene that inhibits collagen synthesis. Compounds capable of regulating the expression of a gene that inhibits collagen synthesis may be useful for modulating a fibrotic process or treating a fibrotic condition.

### FIELD

The present invention is directed, generally, to a method of identifying compounds that modulate collagen synthesis and the use of such compounds to modulate a fibrotic process and/or treat a fibrotic condition. More specifically, the present invention is directed to a method of identifying compounds that regulate the expression of certain genes that inhibit the activity of collagen synthesizing genes.

### BACKGROUND

Fibrotic processes and conditions are generally related to the formation or development of fibrous connective tissue by cells in an organ or tissue. Although fibrotic processes and conditions occur as part of normal tissue formation or repair (e.g., the formation of scar tissue when a wound heals), dysregulation of these processes can lead to altered cellular composition and excess connective tissue deposition that progressively impairs tissue or organ function resulting in a fibrotic condition. An undesirable fibrotic condition is sometimes referred to as fibrosis. One example of an undesirable fibrotic condition is pulmonary fibrosis, which is a disease that occurs when the air sacs of the lungs gradually become replaced by fibrotic tissue. However, not all fibrotic processes or conditions are undesirable. For example, the formation of collagen in skin, which helps provide skin with strength and elasticity, can be desirable in some instances. Thus, identifying compounds and methods useful for modulating fibrotic conditions has long been a goal for pharmaceutical and cosmetic manufacturers.

Research into the genetic underpinning of fibrotic conditions has led to the discovery of new genetic and biomolecular targets. For example, U.S. Pat. No. 10,048,250 describes targets that play a role in the differentiation of macrophages into M2 macrophages, and in particular a suppression of the release or expression of CCL18 and/or CD206. U.S. Publication No. 2013/0209490 describes a method for inhibiting the fibrotic activity of a cell by using a BMP9 or BMP10 antagonist. U.S. Publication No. 2009/0220488 describes a process for treating or preventing scleroderma or other fibrotic disorders by administering an effective amount of a Wnt signaling antagonist. US 2009/0220488 also discloses treating or preventing scleroderma by administering an effective amount of an agent that decreases expression of a gene identified by the researchers as being altered in relation to the expression of scleroderma. However, there is still a need to identify new targets that are involved in fibrotic processes and conditions.

One particular fibrotic process of interest to researchers in the medical and cosmetic skin care fields is the formation of collagen in skin. Collagen is the primary component in the extracellular matrix (ECM) and makes up approximately one-third of the protein in the human body. In human skin, collagen is generated by dermal fibroblasts and then secreted into the ECM, where it aggregates with the existing matrix to form an interlocking mesh of fibrous proteins. Skin quality and appearance depend to a great extent on the properties of the dermis and its extracellular matrix. Failure to maintain appropriate collagen amounts is thought to underlie clinical manifestations of skin aging such as wrinkles, sagginess, and laxity.

While significant research has been done on the collagen synthesis pathway, it is a complex biochemical process, as illustrated in FIG. 1, and there is still much that is not known. In particular, relatively large parts of collagen regulation are not well elucidated, especially regarding signaling pathways that inhibit steps in the collagen synthesis pathway. Thus, it may be desirable to modulate collagen synthesis by identifying and targeting genes that inhibit collagen synthesis rather than the genes directly involved in collagen synthesis. However, it appears from the foregoing publications that past researchers have not adequately explored this possibility.

Accordingly, it would be desirable to understand molecular and cellular processes related to fibrotic conditions and to provide new biomolecular targets for modulating fibrotic processes and conditions. It would also be desirable to provide new methods of identifying compounds that can modulate genetic and/or biomolecular targets related to fibrotic conditions and/or processes. It would further be desirable to identify targets involved in the suppression of genes associated with collagen production in skin.

### SUMMARY

The present disclosure describes novel methods of modulating a fibrotic condition, identifying compounds for modulating a fibrotic condition, and making compositions for modulating a fibrotic condition. In one embodiment, a method of identifying compounds capable of modulating a fibrotic process is disclosed. The method comprises contacting a plurality of immortalized or transformed fibroblasts with a test compound; determining a level of activity of a collagen inhibiting gene of the fibroblasts contacted with the test compound; comparing the level of activity of the collagen inhibiting gene to a control; and identifying the test compound as being capable of modulating a fibrotic process when the activity of the collagen inhibiting gene indicates an upregulation or downregulation of the collagen inhibiting gene relative to the control.

### BRIEF DESCRIPTION OF THE FIGURES

FIG. 1 illustrates some of the steps in the collagen biosynthesis pathway.

FIG. 2 illustrates the process to make the Red-COL1A1 reporter cell line.

FIGS. 3A and 3B illustrate the effect of alpha-ergocryptine on collagen synthesis.

FIGS. 4A-E illustrate the effect of either inhibiting or increasing the levels of collagen regulating genes on collagen levels.

FIG. 5 List of Human Collagen Synthesizing Genes.

FIG. 6 List of Human Collagen Inhibiting Genes.

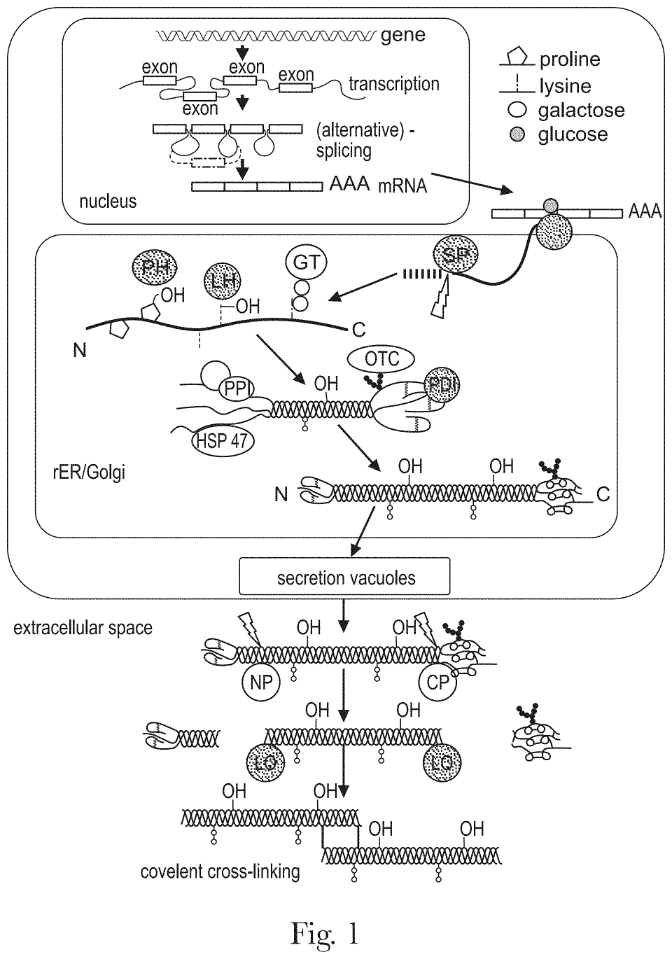

### DETAILED DESCRIPTION

Previous research into fibrotic processes and conditions did not adequately appreciate that there is an unexpectedly large number of genes capable of inhibiting collagen synthesis. These collagen inhibiting genes code for proteins and/or RNA that can inhibit activation of collagen synthesizing genes and/or interfere with activity of the gene products (e.g., proteins) of the collagen synthesizing genes. It has now been discovered that modulating the expression of one or more of these collagen modulating genes and/or the proteins or RNA encoded by these genes can be used to modulate collagen synthesis. Thus, it may be desirable to target the activity of collagen inhibiting genes rather than the collagen synthesizing genes. In particular, it may be desirable to target the activity of genes previously not known to be collagen inhibiting genes.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages are by weight of the corresponding composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

The transcriptional profiles herein can comprise, consist essentially of, or consist of, data related to the genes in a subject gene signature (e.g., in the form of gene identifiers and direction of regulation) as well as other optional components described herein (e.g., metadata). As used herein, “consisting essentially of” means that a transcriptional profile includes data related to the transcription of only select genes from a subject gene signature or gene expression profile, but may also include additional data only if the additional data is not related to the transcription of genes not included in the subject gene signature, and which do not materially alter the basic and novel characteristics of the claimed compositions or methods. As used in the description and the appended claims, the singular forms “a,” “an,” and “the” are intended to include the plural forms as well, unless the context clearly indicates otherwise.

The gene and protein designations disclosed herein correspond to their respective known sequences in the National Institute of Health's genetic sequence database, GenBank®, as of Aug. 23, 2019 and are incorporated herein by reference.

### Definitions

“About” modifies a particular value by referring to a range equal to plus or minus twenty percent (+/−20%) or less (e.g., less than 15%, 10%, or even less than 5%) of the stated value.

“Cosmetic agent” means any substance, as well any component thereof, intended to be rubbed, poured, sprinkled, sprayed, introduced into, or otherwise applied to a mammalian body or any part thereof. Cosmetic agents may include substances that are Generally Recognized as Safe (GRAS) by the US Food and Drug Administration, food additives, and materials used in non-cosmetic consumer products including over-the-counter medications. In some embodiments, cosmetic agents may be incorporated in a cosmetic composition comprising a dermatologically acceptable carrier suitable for topical application to skin. Some non-limiting examples of cosmetic agents or cosmetically actionable materials can be found in: the PubChem database associated with the National Institutes of Health, USA; the Ingredient Database of the Personal Care Products Council; and the 2010 International Cosmetic Ingredient Dictionary and Handbook, 13th Edition, published by The Personal Care Products Council; the EU Cosmetic Ingredients and Substances list; the Japan Cosmetic Ingredients List; the Personal Care Products Council, the SkinDeep database; the FDA Approved Excipients List; the FDA OTC List; the Global New Products Database (GNPD); and from suppliers of cosmetic ingredients and botanicals.

“Fibroblast” means a connective-tissue cell of mesenchymal origin that secretes proteins, especially molecular collagen, to form the extracellular fibrillar matrix of connective tissue.

“Fibrotic condition” refers to a biological condition resulting from an excessive or insufficient amount of collagen in a tissue. One example of a fibrotic condition is thinner, less elastic skin resulting from an age-induced reduction in collagen production. Another example of a fibrotic condition is pulmonary fibrosis.

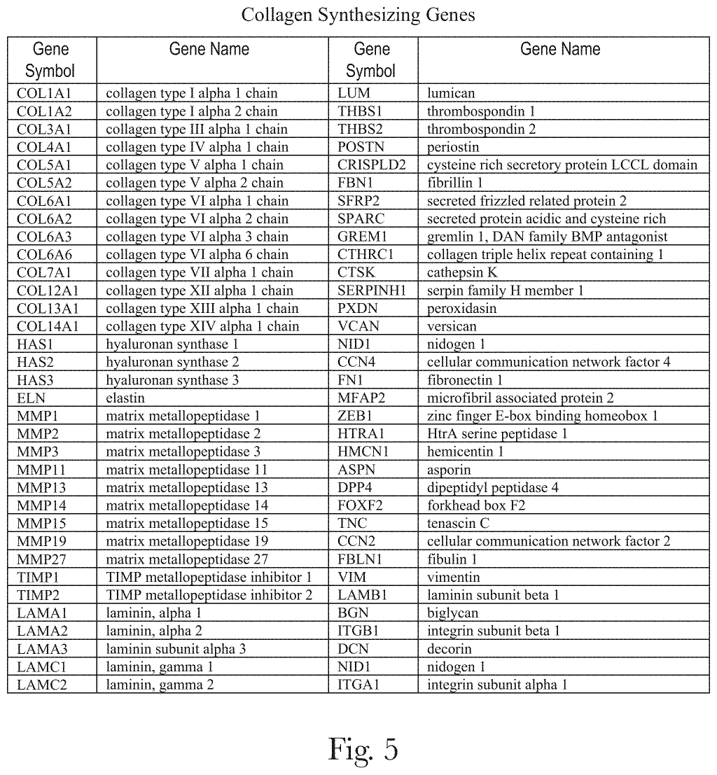

“Fibrotic process” refers to a biological process related to the synthesis of collagen and/or incorporation of collagen into the ECM. An example of a fibrotic process is the expression of one or more of the genes in FIG. 5 as it relates to collagen synthesis.

“Gene expression profiling” and “gene expression profiling experiment” mean a measurement of the expression of multiple genes in a biological sample using any suitable profiling technology. For example, the mRNA synthesis of thousands of genes may be determined using microarray techniques. Other emerging technologies that may be used include RNA-Seq or whole transcriptome sequencing using NextGen sequencing techniques.

“Gene product” means an RNA or protein resulting from the expression of a gene.

“Immortalized fibroblasts” are fibroblasts that, due to mutation, evade normal cellular senescence and instead can keep undergoing division indefinitely, thereby allowing them to be grown for prolonged periods in vitro.

“Microarray” means any ordered array of nucleic acids, oligonucleotides, proteins, small molecules, large molecules, and/or combinations thereof on a substrate that enables gene expression profiling of a biological sample. Some non-limiting examples of microarrays are available from Affymetrix, Inc.; Agilent Technologies, Inc.; Ilumina, Inc.; GE Healthcare, Inc.; Applied Biosystems, Inc.; and Beckman Coulter, Inc.

“Parental cell,” when referring to immortalized or transformed fibroblasts herein, means the cell line that was modified (i.e., immortalized or transformed) to produce immortalized or transformed fibroblasts.

“Positive control” refers to a compound (or mixture of compounds) that has a known effect on a particular fibrotic process or condition and/or modulates the expression of one or more genes associated with the fibrotic process or condition in a known direction.

“Safe and effective amount” means an amount of a compound or composition sufficient to significantly induce a positive benefit (e.g., an increase in collagen synthesis by dermal fibroblasts), including independently or in combinations the benefits disclosed herein, but low enough to avoid serious side effects, i.e., to provide a reasonable benefit to risk ratio, within the scope of sound judgment of the skilled artisan.

“Skin” means the outermost protective covering of mammals that is composed of cells such as keratinocytes, fibroblasts and melanocytes. Skin includes an outer epidermal layer and an underlying dermal layer. Skin may also include hair and nails as well as other types of cells commonly associated with skin, such as, for example, myocytes, Merkel cells, Langerhans cells, macrophages, stem cells, sebocytes, nerve cells and adipocytes.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity; improve skin hydration; improve skin condition; and improve cell metabolism).

“Transformed fibroblasts” are fibroblasts that, due to mutation, undergo a state of unregulated growth in vitro and exhibit the hallmark characteristics of cancer cells.

Gene modulation generally involves increasing or decreasing the amount of gene expression products produced by a gene. Gene modulation is also sometimes referred to as gene regulation, where “upregulating” the gene means promoting and/or increasing the production of the gene product and “downregulating” the gene means inhibiting and/or decreasing the production of the gene product. Like most genes, genes involved in the production of collagen can be modulated at any step in the gene expression pathway (e.g., from DNA-RNA transcription to post-translational modification of a protein) by a variety of different mechanisms used by cells to increase or decrease the production of specific genes. Human Genes of interest that directly contribute to collagen synthesis are referred to herein as “collagen synthesizing genes” and are shown in FIG. 5. It is to be appreciated that subsets of the genes provided in FIG. 5, which are not specifically listed, may be particularly useful in the methods herein.

Collagen consists of protein strands wound together to form triple-helices of elongated fibrils. Depending upon the degree of mineralization, collagen tissues may be rigid (bone), compliant (tendon), or have a gradient from rigid to compliant (cartilage). There are numerous different types of collagen found in the human body, with Type I being the most common type found in skin. An exemplary description of the different types of collagens can be found in K. Gelse, et al., (2003) “Collagens—structure, function, and biosynthesis;” Advanced Drug Delivery Reviews 55; 1531-1546. Dysfunctional overproduction of collagen is the primary cause of a variety of fibrotic diseases such as scleroderma and pulmonary fibrosis. Conversely, the underproduction of collagen is responsible for at least some characteristics of aging skin (e.g., sagginess, loss of elasticity, thinning, and wrinkle formation).

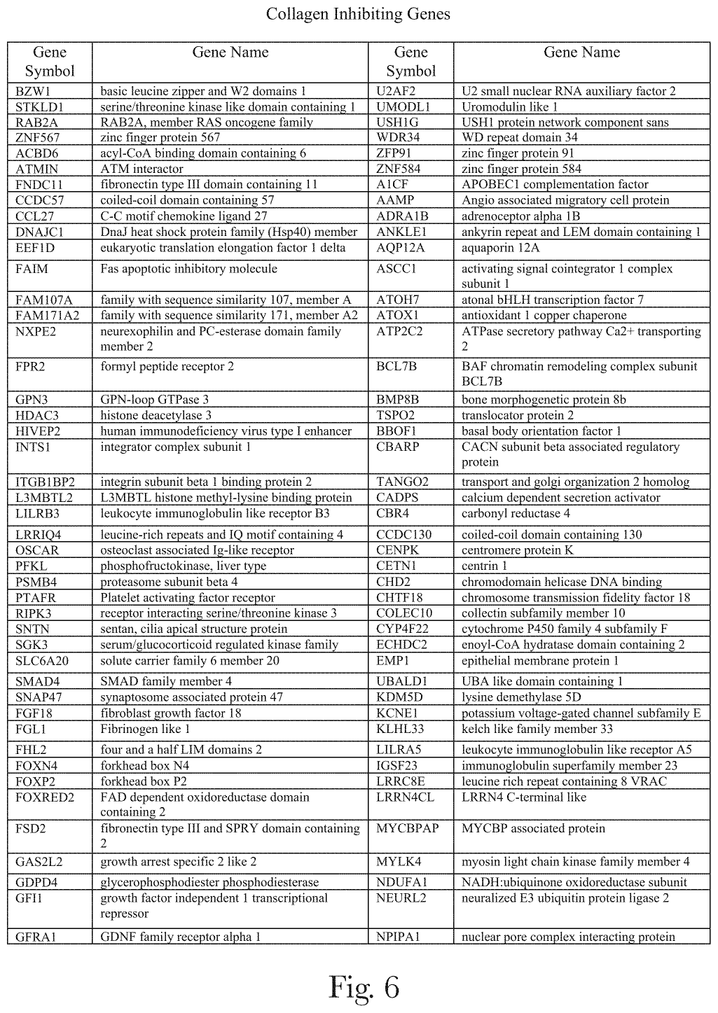

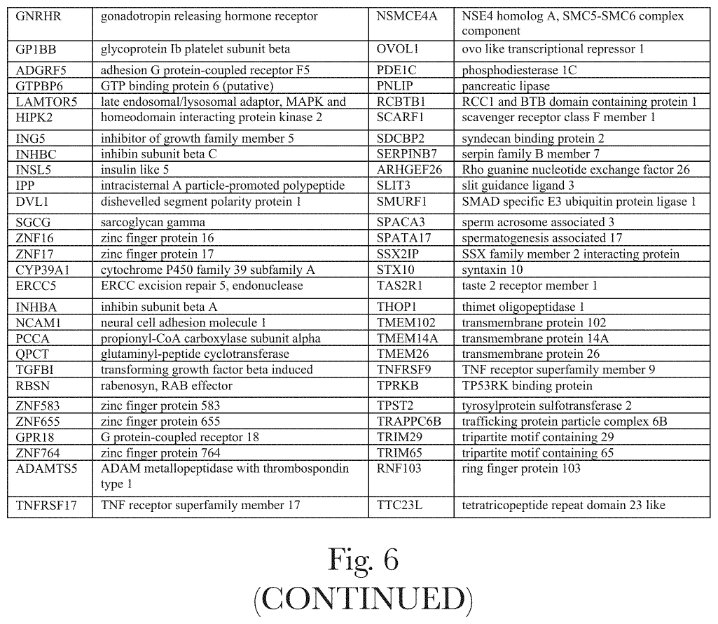

Upregulating the expression of collagen synthesizing genes typically leads to an increase in collagen production. Conversely, inhibiting expression of these collagen synthesizing genes should lead to a decrease in collagen production. Enhancing or inhibiting the activity of the gene expression products of collagen synthesizing genes may also result in higher or lower amounts of collagen, respectively. Additionally, there is a relative abundance of genes that code for products that inhibit collagen synthesis, for example, by suppressing activation of a collagen synthesizing gene and/or interfering with a gene expression product of a collagen synthesizing gene. Due to the complexity of trying to modulate collagen synthesis directly (e.g., by directly modulating gene expression of collagen synthesizing genes or inhibiting the activity of their gene products), it may be desirable to modulate the expression of one or more collagen inhibiting genes rather than modulating the expression of one or more collagen synthesizing genes. For example, it may be easier to increase the amount of collagen in skin by knocking down a collagen inhibiting gene (e.g., via RNA silencing) rather than upregulating a collagen synthesizing gene. As used herein, “collagen inhibiting genes” refers to Human Genes of interest that inhibit collagen synthesis and are listed in FIG. 6. The collagen inhibiting genes share the common function of inhibiting collagen synthesis, and thus can be used, collectively or individually, to modulate the expression of collagen synthesizing and/or the activity of collagen synthesizing gene expression products. It is to be appreciated that subsets of the genes provided in FIG. 6, which are not specifically listed, may be particularly useful in the methods herein and are contemplated by the present invention. For example, the method may utilize a subset of 2, 3, 4, 5, 10, 15, or even 20 or more of the collagen inhibiting genes.

### Identifying a Compound that Modulates a Fibrotic Process

The methods herein involve identifying test compounds capable of modulating a fibrotic process (“fibrotic agent”) and/or evaluating the ability of a compound to modulate a fibrotic process or treat a fibrotic condition. The present method may be used to identify fibrotic agents that modulate the expression of a collagen synthesizing gene, modulate the expression of a collagen inhibiting gene, and/or interfere with the activity of a gene product of a collagen synthesizing gene or collagen inhibiting gene. In some embodiments, the present method may be employed as a high throughput screening method (i.e., a screening method in which at least 25 test compounds can be tested simultaneously). For example, the present method may be used to screen up to 1,000 test compounds simultaneously (e.g., 50, 100, 200, 500, or 750 or more). In some embodiments, the method involves determining the amount of change in the rate and/or amount of collagen synthesis relative to a control. The change in collagen synthesis may be determined by protein quantitation, reporter gene activity, and/or gene transcriptomic analysis.

Some non-limiting examples of test compounds for use in the present method are small molecules, nucleic acids (e.g., small interfering RNA, micro RNA, and small activating RNA), antibodies, plant extracts, vitamins, minerals, cosmetic agents, and other compounds for which collagen modulating ability is desired to be known. In some instances, it may be desirable to determine whether a test compound can modulate the expression of two or more collagen inhibiting genes (e.g., between 3 and 100 genes, 5 and 50 genes, 10 and 30 genes, or even between 12 and 20 genes). For example, it may be desirable to determine whether a test compound can modulate the expression of two or more collagen inhibiting genes that share a common mechanism of action, share a common biochemical pathway, or belong to the same gene family or superfamily (e.g., ZFP91, ZNF16, ZNF17, and ZNF584, or FAM107A and FAM171A2, or CCDC57 and CCDC 130). In another example, it may be desirable to determine whether a test compound can modulate the expression of a collagen inhibiting gene whose gene product is druggable. “Druggable” means that a biological target such as a target protein is known or predicted to bind with high affinity to a drug or antibody, which alters the function of the target protein. An example of a druggable target may a protein encoded by a collagen inhibiting gene.

In some embodiments, the present method involves contacting a plurality of immortalized or transformed fibroblasts with a test compound; determining the level of activity of a collagen inhibiting gene of the fibroblasts; comparing the level of activity of the collagen inhibiting gene to a control; and identifying the test compound as a fibrotic agent which is capable of modulating a fibrotic process when the measured activity indicates an upregulation or downregulation of the collagen inhibiting gene relative to the control. Methods of determining the level of activity of a collagen inhibiting gene are described in more detail below. In some embodiments, the method may also involve incorporating a fibrotic agent into a composition and/or administering the fibrotic agent to a person in need of treatment.

The fibroblasts used in the present method are not particularly limited and can include commercially available cell lines (e.g., TERT fibroblasts from ATCC; Monassas, Va.), ex vivo fibroblasts obtained from human donors, and/or modified versions of these (e.g., immortalized, transformed, or modified to include a reporter gene). The fibroblasts used in the present methods can be cultured using conventional methods of culturing cells lines of this type to provide a test sample. The test sample is contacted with a test compound for a sufficient amount of time to determine whether the test compound can modulate collagen synthesis. The test compound may be any compound that is safe for human use (e.g., via ingestion, inhalation, topical application, and/or injection) and may be suitable for use in a pharmaceutical composition (e.g., a composition only available through a prescription from a licensed professional), a cosmetic composition (e.g., an over-the-counter product such as skin lotion), or a cosmeceutical composition (e.g., an over-the-counter product such as a skin lotion that includes an active ingredient known to provide a particular benefit). The test compound may be in pure form or it may be mixed with other ingredients to facilitate contact with the fibroblasts in the test sample.

In some instances, it may be desirable to determine collagen synthesis activity by measuring the activity of one or more collagen synthesizing genes and/or collagen inhibiting genes via a reporter gene inserted downstream of an endogenous promoter for the gene of interest. The reporter gene may encode for a protein that emits at a defined fluorescent wavelength when excited by a specific wavelength range. In some instances, it may be desirable to use an exogenous collagen synthesizing gene and/or collagen inhibiting gene promoter linked to a reporter gene. For example, a collagen synthesizing gene and/or collagen inhibiting gene promoter may be cloned into a plasmid, linked to a reporter construct and transfected into an immortalized/transformed fibroblast cell line. The amount of fluorescent protein present in the fibroblast sample can be quantitated and directly correlated to the activity of the gene of interest. The fluorescent protein may be quantitated using a suitable fluorescence spectroscopy technique (e.g., using a fluorometer according to the manufacturer's instructions). The fluorescent protein used herein, or the gene that codes for it, is not particularly limited and includes green fluorescent proteins (“GFP”) and red fluorescent proteins (“RFP”). Some non-limiting examples of RFPs are mCherry, mStrawberry, mOrange, and dTomato. In a more specific example, it may be desirable to use an immortalized or transformed fibroblast cell line modified via a gene editing technique (e.g., zinc finger nuclease, transcription activator-like effector nucleases (TALEN), or clustered regularly interspaced short palindromic repeats (CRISPR) to include a nuclear-localizing signal mCherry gene (“NLS-mCherry”) that codes for the mCherry RFP when the gene of interest (e.g., COL1A1) is activated.

In some instances, it may be desirable to use transcriptomic analysis to measure gene activity of cells contacted with a test compound relative to a control. A test compound is identified as a fibrotic agent capable of modulating collagen synthesis when the transcriptional profiles of the test sample and control sample, relative to one another, correspond to a change in expression of at least one of the genes in FIG. 5 or FIG. 6 in a direction indicative of the desired change in collagen production. For example, if an increase in collagen production is desired, then the test sample should show an upregulation in one or more genes from FIG. 5 and/or a downregulation of one or more genes from FIG. 6. In some instances, messenger ribonucleic acid (“mRNA”) encoded by one or more genes of interest in a gene signature may be measured and compared to a control. In some instances, the mRNA may be reverse transcribed and the corresponding complementary DNA (“cDNA”) measured. Any suitable quantitative nucleic acid assay may be used. For example, conventional quantitative hybridization, Northern blot, and polymerase chain reaction procedures may be used for quantitatively measuring the amount of an mRNA transcript or cDNA in a biological sample. Optionally, the mRNA or cDNA may be amplified by polymerase chain reaction (PCR) prior to hybridization. The mRNA or cDNA sample is then examined by, e.g., hybridization with oligonucleotides specific for mRNAs or cDNAs encoded by the one or more of the genes of interest (e.g., collagen inhibiting genes), optionally immobilized on a substrate (e.g., an array or microarray). Binding of the nucleic acid to the oligonucleotide probes specific for the gene of interest allows identification and quantification of the expression level of that gene. Suitable examples of transcriptomic methods of quantifying gene expression are disclosed in U.S. Pat. Nos. 9,434,993; 10,036,741; and 10,282,514.

In some instances, the test compound may be dissolved in a suitable vehicle such as dimethyl sulfoxide (DMSO), water, or an aqueous/organic combination solvent such as ethanol/water. After exposure to the test compound and/or control, mRNA can be extracted from the test cells and reference cells. The mRNA extracted from the cells may, optionally, be reverse transcribed to cDNA and marked with fluorescent dye(s) (e.g., red and green if a two-color microarray analysis is to be performed). In some instances, the cDNA samples may be prepped for a one-color microarray analysis, and a plurality of replicates may be processed if desired. The cDNA samples may be co-hybridized to the microarray comprising a plurality of probes (e.g., tens, hundreds, or thousands of probes). In some embodiments, each probe on the microarray has a unique probe set identifier. The microarray is scanned by a scanner, which excites the dyes and measures the amount fluorescence. A computing device analyzes the raw images to determine the amount of cDNA present, which is representative of the expression levels of a gene. The scanner may incorporate the functionality of the computing device. Typically, gene expression data collected by the system may include: i) up-regulation of gene expression (e.g., greater binding of the test material (e.g., cDNA) to probes compared to reference material (e.g., cDNA)), ii) down-regulation of gene expression (e.g., reduced binding of the test material (e.g., cDNA) to probes than the test material (e.g., cDNA)), iii) non-fluctuating gene expression (e.g., similar binding of the test material (e.g., cDNA) to the probes compared to the reference material (e.g., cDNA)), and iv) no detectable signal or noise. The up- and down-regulated genes may be referred to as “differentially expressed.” Differentially expressed genes may be further analyzed and/or grouped together (e.g., via known statistical methods) to identify genes that are representative of a fibrotic condition or biological response to a test compound.

In some instances, it may be desirable to determine collagen synthesis activity by protein quantitation, for example, using a conventional assay such as an enzyme-linked immunosorbent assay (ELISA), a Western blot, mass spectrometry, a UV absorption, a bicinchoninic acid, a Bradford assay, a Kjeldahl assay, or a Folin-Lowry assay. Other non-limiting examples of methods for quantitating collagen are described in L. C. U. Junqueira, et al., (1979) “A Simple and Sensitive Method for the Quantitative Estimation of Collagen;” Analytical Biochemistry 94; 96-99; and R. F. Diegelmann, et al., (1990) “A Microassay to Quantitate Collagen Synthesis by Cells in Culture;” Analytical Biochemistry 186 (2); 296-300.

### Compositions Containing a Fibrotic Agent

Once a compound is identified as a fibrotic agent, it may be incorporated into a composition and administered to a person in need of treatment. Of course, it is to be appreciated that, in some instances, it may be desirable to administer the fibrotic agent in a pure form (i.e., undiluted or free of a carrier) to a person in need of treatment. The fibrotic agent may be mixed with a suitable carrier and other optional ingredients, e.g., using conventional formulation and processing techniques, to provide a composition suitable for administering to a person. Optional ingredients included in the present compositions are not particularly limited as long as they do not unacceptably alter the ability of the composition to modulate a fibrotic process such as collagen synthesis. The optional components, when present, should be suitable for use with human tissue without undue toxicity, incompatibility, instability, allergic response, and the like. The optional ingredients may be present at 0.0001% to 50% (e.g., 0.001% to 20% or even 0.01% to 10%). The amounts listed herein are only to be used as a guide, as the optimum amount of the optional ingredients used in a composition will depend on the specific ingredient selected since their potency and/or function can vary considerably.

The form of the composition should be tailored for the desired administration route of the fibrotic agent (e.g., topical application, oral ingestion, or injection). For example, the composition may be in the form of a solution, suspension, dispersion, emulsion, powder, tablet, capsule, lotion, cream, gel, toner, spray, aerosol, ointment, cleansing liquid wash, solid bar, shampoo, hair conditioner, paste, foam, powder, mousse, shaving cream, wipe, strip, patch, wound dressing, adhesive bandage, hydrogel, film-forming product, facial and skin mask (with and without insoluble sheet). The composition may be provided in a package sized to store a sufficient amount of the composition for a treatment period.

In some instances, a composition herein may be formulated for topical administration, e.g., to the scalp, skin, or mucosa of a person, by mixing the fibrotic agent with a dermatological acceptable carrier. Such compositions may also include one or more optional ingredients of the kind commonly included such compositions. “Dermatologically acceptable carrier” means that a carrier that is suitable for topical application to keratinous tissue, has good aesthetic properties, is compatible with the ingredients in the composition, and will not cause any unreasonable safety or toxicity concerns. The dermatologically acceptable carrier may be present at 1% to 95% (e.g., 10% to 90%, 30% to 70%, 50% to 60%) by weight of the composition. The carrier may be aqueous or anhydrous. For example, suitable carriers may include water, water miscible solvents, and oils. Suitable water miscible solvents include monohydric alcohols, dihydric alcohols, polyhydric alcohols, glycerol, glycols, polyalkylene glycols such as polyethylene glycol, and mixtures thereof. Suitable oils include silicones, hydrocarbons, esters, amides, ethers, and mixtures thereof. The oils may be volatile or nonvolatile.

Optional ingredients that may be added to a composition intended for topical application to skin can include, without limitation, anti-acne actives, desquamation actives, anti-cellulite agents, chelating agents, flavonoids, tanning active, non-vitamin antioxidants and radical scavengers, hair growth regulators, anti-wrinkle actives, anti-atrophy actives, minerals, phytosterols and/or plant hormones, N-acyl amino acid compounds, antimicrobial or antifungal actives, and other useful skin care actives. Some non-limiting examples of skin care actives that may be suitable for use in the present composition are described in U.S. Publication Nos. 2006/0275237 and US 2004/0175347 and The International Cosmetic Ingredient Dictionary and Handbook, Thirteenth Edition.

In some instances, the optional ingredients for use in the present compositions may include known anti-fibrotic actives to inhibit collagen production. Some non-limiting examples of anti-fibrotic actives are disclosed in U.S. Publication No. 2013/0209490; U.S. Pat. No. 7,026,283; and Wynn, et al., Journal Clin. Invest., Vol 117 Number 3, March 2007, p 524. In some instances, the composition may optionally include fibrotic actives that promote collagen production (“pro-fibrotic actives”), for example, by acting on the same or similar biochemical pathways as the anti-fibrotic actives, but with opposite effect.

The present compositions may be prepared by conventional methods for making such compositions. These methods typically involve mixing of ingredients in one or more steps to a relatively uniform state, with or without heating, cooling, application of vacuum, and the like. Typically, emulsions are prepared by first mixing the aqueous phase materials separately from the fatty phase materials and then combining the two phases as appropriate to yield the desired continuous phase. The compositions are preferably prepared such as to optimize stability (physical stability, chemical stability, photostability, etc.) and/or delivery of active materials.

### Methods of Use

Generally, the fibrotic agents identified by the present methods may be administered in accordance with conventional methods of using compositions of the type. Compositions comprising an effective amount of a fibrotic agent can be used to modulate a fibrotic process and/or treat a fibrotic condition by administering the composition to person in need of treatment. A person in need of treatment is one who exhibits symptoms of a fibrotic condition or who is diagnosed with a fibrotic condition. Of course, it is to be appreciated that in some instances, a person who does not exhibit symptoms of a fibrotic condition or has not been diagnosed with a fibrotic condition may still desire treatment. For example, a user may wish to target a portion of skin that does not exhibit a symptom of collagen depletion (e.g., wrinkles and saggy skin), but is known to develop symptoms of the condition (e.g., skin surfaces that are typically not covered by clothing, such as facial skin). The compositions herein may be administered in any suitable manner that is effective to treat the fibrotic condition and/or modulate the fibrotic process.

Compositions containing an effective amount of a fibrotic agent for modulating a fibrotic process may be administered once a day, twice a day, or on a more frequent daily basis, during a treatment period. The treatment period is ideally of sufficient time for the fibrotic agent to provide the desired benefit. For example, the treatment period may be of sufficient time for the fibrotic agent to provide a noticeable and/or measurable improvement in a fibrotic condition or change in a fibrotic process. The treatment period may last for at least 1 week (e.g., about 2 weeks, 4 weeks, 8 weeks, or even 12 weeks). In some instances, the treatment period will extend over multiple months (i.e., 3-12 months) or multiple years. In some instances, a composition containing an effective amount of a fibrotic agent may be administered most days of the week (e.g., at least 4, 5 or 6 days a week), at least once a day or even twice a day during a treatment period of at least 2 weeks, 4 weeks, 8 weeks, or 12 weeks.

### EXAMPLES

Non-limiting example of various aspects of the methods described herein are provided below. The examples are given solely for the purpose of illustration and are not intended to be construed as limiting the invention, as many variations thereof are possible.

### Example 1

This example describes a novel reporter system (“Red-COL1A1”) that can be used to determine the level of activity of a gene. The Red-COL1A1 reporter system was used to identify collagen inhibiting genes of interest.

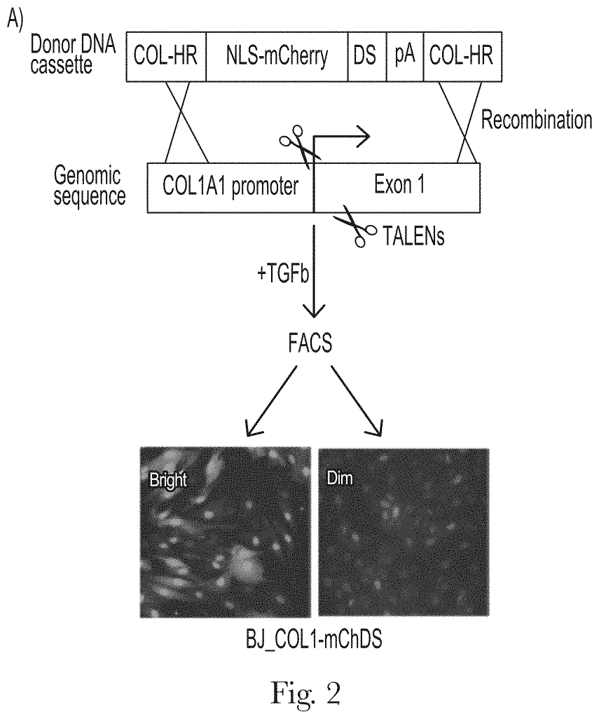

In this example, RNA interference (“RNAi”) screening was used to identify genes that appear to inhibit collagen synthesis. Immortalized human skin fibroblasts (BJhTERT, available from ATCC, Manassas, Va. were modified to place an mCherry reporter gene downstream of the endogenous promoter for COL1A1 using zinc nucleases (TALENs). COL1A1 is well known for its role in the synthesis of Type I collagen, sometimes referred to as collagen I, which is a fibrillar collagen that accounts for over 90% of the collagen found in the human body. The reporter gene codes for the mCherry RFP, which can be quantitated using known fluorescent protein detection techniques. Transforming growth factor beta (“TGFβ”), a well-known COL1A1 activating cytokine, was used in a fluorescence-activated cell sorting (“FACS”) technique to select for a cell line with low endogenous expression and high levels of inducible expression of the mCherry gene. A summary of the process used to develop the cell line is illustrated in FIG. 2.

A genome wide screen was performed on approximately 22,000 genes using the Red-COL1A1 reporter cell line to identify genes with the ability to regulate collagen expression. The genome wide screen involved performing RNAi screening on the Red-COL1A1 cell line according to the following method. 2.5 μl of 500 nM siRNA (Dharmacon) was robotically pre-printed onto black-walled, 384-well microplates (Greiner #781091) with Agilent Technologies Velocity 11. Reverse siRNA transfection for each well containing a test sample was performed by pre-mixing 0.2 μl Lipofectamine RNAiMAX with 7.3 μl Gibco Opti-MEM Reduced Serum Medium for 5 min and dispensing the mixture into the pre-printed siRNA assay plates to form RNA-lipid complexes. All dispensing into 384-well plates was performed with Thermo Scientific Multidrop Combi Reagent Dispenser. Complexes were allowed to form for 20 min before dispensing 3,000 Red-COL1A1 cells into each well. At 3 days post-transfection, TGFβ was added at a final concentration of 10 ng/mL to selected control wells (positive control). The pilot screen was performed in duplicate. After 6 days of knockdown, cells were fixed using 4% paraformaldehyde containing 2% sucrose for 15 min, washed once with D-PBS, and stained for 10 min with Hoechst 33342 diluted in D-PBS (1:10,000). The cells were then washed two more times for 5 min with D-PBS before high-throughput imaging.

Automated image acquisition and analysis was used to quantitate the fluorescent protein. Four sites (each imaged at two excitation wavelengths of 405 and 561 nm) per well were acquired sequentially at 10× magnification on an IMAGEXPRESS brand micro high-content imaging software from Molecular Devices. Images were analyzed using METAXPRESS brand high-content image analysis software from Molecular Devices. The percentage of cells with positive nuclear-localizing mCherry signal above background was determined using the “Translocation-Enhanced” module. The average fold-change percentage of positive mCherry cells for each sample well was normalized with reference to mean of siNT (n=8) in the same plate. Raw data were processed and analyzed using SCREENSIFTER brand RNAi data-screening software from BMC Bioinformatics, and the average ±SD for screen replicates were determined. The threshold for positive hits (>2) was derived using the derivative method available in the data-screening software. Similarly, the average cell count per sample well was normalized to the mean cell count in siNT wells in the same plate, with the threshold arbitrarily set to 80%.

The genome wide screen resulted in the identification of 322 genes (Z-score>10) that inhibit collagen production as demonstrated by their ability to increase m-cherry expression in Red-COL1A1 cells when knocked down. To exclude off-target RNAi effects, deconvoluted RNAi screens were performed in which individual siRNAs against each gene were tested against the 297 genes that were available for purchase from Dharmacon. The screen was performed in 2 replicates each with technical duplicates. Individual siRNA tends to elicit a less potent effect compared to pooled siRNAs. Therefore, using 2.5-fold as a threshold signal, 146 of the 322 genes identified as inhibiting collagen production were confirmed by at least two independent siRNAs. Evaluation of the effect of knockdown of these 146 genes on collagen protein production demonstrated that the induction of both the m-cherry protein and the collagen 1A1 protein in the Red-COL1A1 BJ fibroblast cell line. Confirmation was performed in additional fibroblast cell lines and the method was validated via Western Blot analysis and quantitative reverse-transcription polymerase chain reaction (qRT-PCR). The collagen inhibiting genes identified in this test are set forth in FIG. 6.

### Example 2

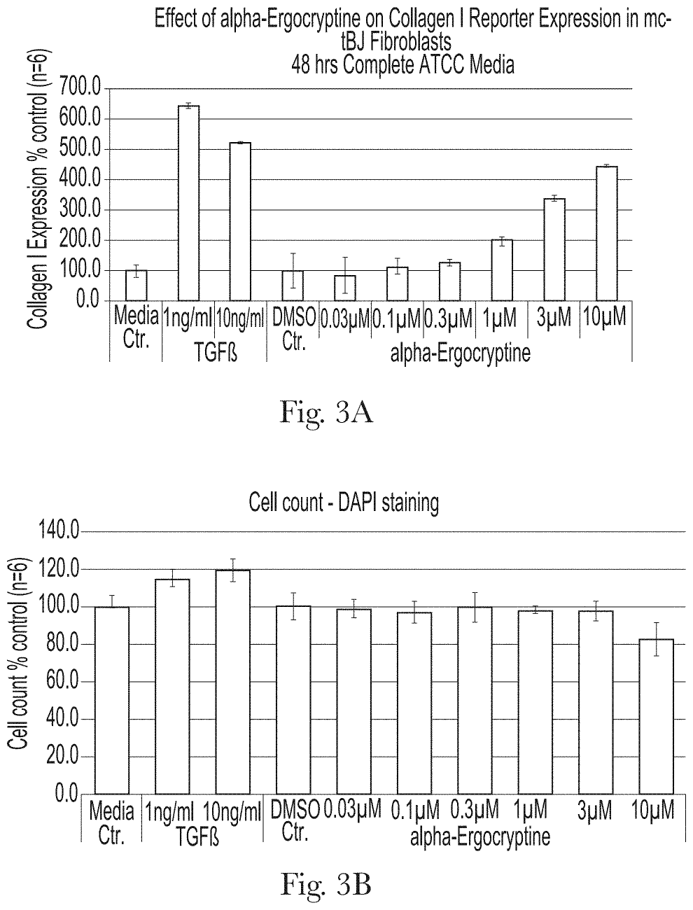

This example demonstrates that collagen synthesizing gene expression can be increased by decreasing the activity of a protein coded by a collagen inhibiting gene. To demonstrate that small molecule inhibition of the proteins of the genes listed in FIG. 6 has a similar effect to that of siRNA knockdown of the expression of these genes, an antagonist of the ADRA1B receptor protein was selected for evaluation of its effect on COL1A1 expression. A summary of the results is illustrated in FIGS. 3A and 3B, which show that increasing doses of the ADRA1B antagonist alpha-ergocryptine increased COL1A1 expression at non-toxic doses. FIG. 3A shows that increasing concentrations of the ADRA1B antagonist alpha-ergocryptine increases the amount of fluorescence resulting from increased expression of the mCherry reporter construct located downstream of the endogenous COL1A1 promoter in immortalized tertBJ fibroblasts cells (see FIG. 2). This increased fluorescence directly correlates to increased collagen synthesis. The results of the positive control (TGFβ), the vehicle control for the TGFβ (Media Control), and the vehicle control for the alpha-ergocryptine (DMSO control) are also illustrated in FIG. 3A. As shown in FIG. 3B, increasing concentrations of alpha-ergocryptine were not toxic to the immortalized tertBJ fibroblasts cells as determined by cell counts. Cell count was determined by staining the nuclei with 4′,6-diamidino-2-phenylindole (DAPI), which is a fluorescent stain that binds strongly to adenine-thymine rich regions in DNA. DAPI staining is a well-known technique for determining cell count in fluorescence microscopy.

### Example 3

This prophetic example demonstrates the ability of the present method to identify fibrotic agents that modulate collagen synthesis using a transcriptional analysis technique. In this example, a suitable microarray (e.g., an Affymetrix GeneChip® or the like) containing probes for the genes in

FIG. 6 is used to identify changes in gene expression for the collagen inhibiting genes. Individual experiments (referred to as batches) generally include 30 to 96 samples analyzed using the selected microarray. Each batch contains 6 replicates of the vehicle control (e.g., DSMO), 2 replicate samples of a positive control that gives a strong reproducible effect in the cell type used (e.g., TGF-β), and samples of the test compound. Replication of the test compound is done in separate batches to compensate for batch effects. In vitro testing is performed in 6-well plates to provide sufficient RNA for GeneChip® analysis (2-4 μm total RNA yield/well).

Human immortalized fibroblasts (e.g., BJ cell line from ATCC) are cultured in Eagle's Minimal Essential Medium (ATCC) supplemented with 10% fetal bovine serum (HyClone, Logan, Utah) in normal cell culture flasks and plates (Corning, Lowell, Mass.). The cells are incubated at 37° C. in a humidified incubator with 5% CO2. Twenty-four hours prior to exposure, the cells are trypsinized from T-75 flasks and plated into 6-well plates in basal growth medium. At t=0 the media is removed and replaced with the appropriate dosing solution as per the experimental design. Dosing solutions are prepared the previous day in sterile 4 ml Falcon snap cap tubes. Pure test materials may be prepared at a concentration of 1-200 μM, and botanical extracts may be prepared at a concentration of 0.001 to 1% by weight of the dosing solution. After 6 to 24 hours of chemical exposure, cells are viewed and imaged. The wells are examined with a microscope before cell lysis and RNA isolation to evaluate for morphologic evidence of toxicity. If morphological changes are sufficient to suggest cytotoxicity, a lower concentration of the test compound is tested. Cells are then lysed with 350 μl/well of RLT buffer containing β-mercaptoethanol (Qiagen, Valencia, Calif.), transferred to a 96-well plate, and stored at −20° C.

RNA from cell culture batches is isolated from the RLT buffer using Agencourt® RNAdvance Tissue-Bind magnetic beads (Beckman Coulter) according to manufacturer's instructions. 1 μg of total RNA per sample is labeled using Ambion Message Amp™ II Biotin Enhanced kit (Applied Biosystems Incorporated) according to manufacturer's instructions. The resultant biotin labeled and fragmented cRNA is hybridized to the selected microarray (e.g., an Affymetrix HG-U133A 2.0 GeneChip®), which is then washed, stained and scanned using the protocol provided by the manufacturer (e.g., Affymetrix). A statistical analysis (e.g., t-test) is conducted on the microarray data to identify collagen inhibiting genes that are regulated by the test compound (either upregulated or downregulated) in a statistically significant way (e.g., p-value ≤0.1, 0.05, or 0.01), and the test compound is identified as being capable of modulating a fibrotic process when at least one of collagen inhibiting genes is upregulated or downregulated.

### Example 4

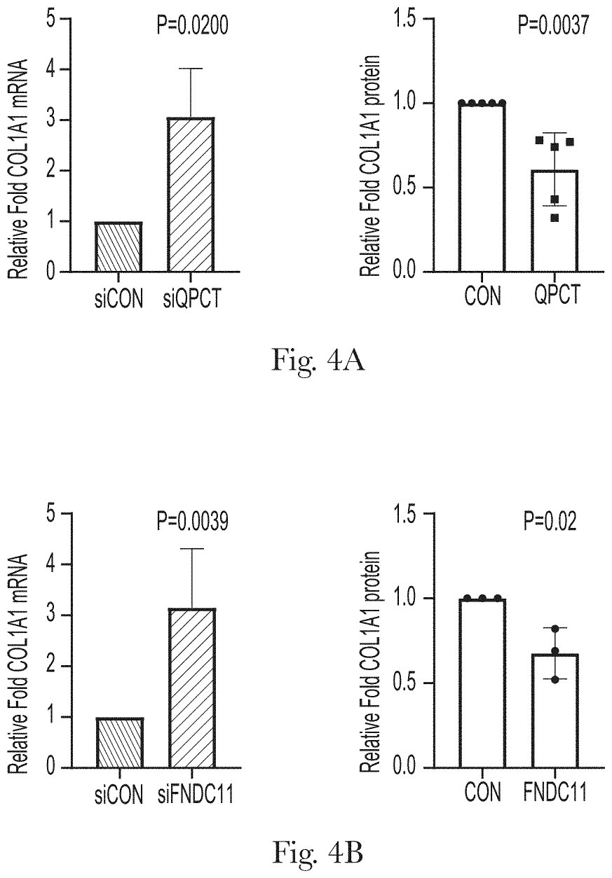

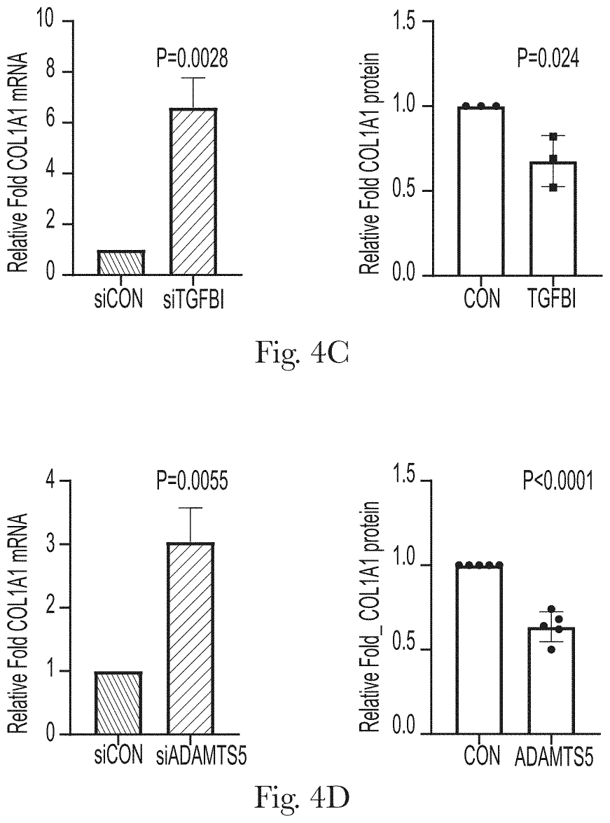

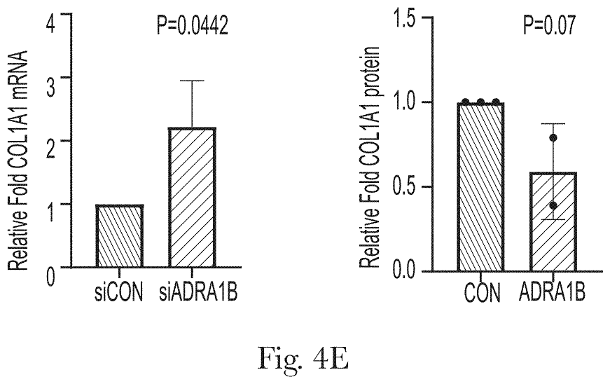

This example demonstrates that collagen synthesizing gene expression can be increased by decreasing the levels of mRNA and protein (Levels of what -mRNA/expression?) of the collagen inhibiting genes and that collagen synthesizing gene expression can be decreased by increasing the levels mRNA and protein of collagen inhibiting genes. To demonstrate that reducing the mRNA levels of genes listed in FIG. 6, and thus proteins, increases the expression of COL1A1 mRNA, the expression of 5 genes including QPCT, FNDC11, TGFBI, ADAMTS5, ADRA1B were reduced using siRNA specific for each of these genes as described in Example 1. A summary of the results is illustrated in FIGS. 4A-E, which show that knocking down the expression of QPCT, FNDC11, TGFBI, ADAMTS5, ADRA1B mRNA using siRNA specific for these genes individually increased COL1A1 mRNA. In contrast, increasing the levels of QPCT, FNDC11, TGFBI, ADAMTS5, ADRA1B proteins by overexpressing these proteins in fibroblasts reduced the levels of COL1A1 protein. Transfection of siRNA into fibroblasts -siRNAs transfections were performed with RNAiMAx (Invitrogen-Thermo Fisher Scientific, Waltham Mass.) in serum free OPTIMEM according to manufacturer instructions. Briefly, 2 μL RNAiMax reagent complexed with the indicated siRNAs (Dharmacon Inc., Lafayette Colo.; Thermo Fisher Scientific) in OPTIMEM for 20 minutes. The reaction mix was added to 24-well plates with 30,000 trypsinised cells. All transfections performed with siRNAs at final concentration of 25 nM. Graphical Nomenclature. siCON—control siRNA; siQPCT, siFNDC11, siTGFBI, siADAMTS5, siADRA1B—siRNA specific for each of these genes. Overexpression of proteins by electroporation—30 ug of plasmids were electroporated into 800,000 BJ-Tert fibroblasts with Neon® Transfection system. Electroporation was performed at 1600 V 10 ins 2 pulses settings using the P100 tip. Cells were plated into 6 well plates and incubated for 48 hours prior to protein and quantification. CON—control expression plasmid without a protein insert; QPCT, FNDC11, TGFBI, ADAMTS5, ADRA1B—expression plasmid containing the indicated proteins.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of identifying fibrotic agents capable of modulating a fibrotic process, comprising:
a) contacting a plurality of immortalized or transformed fibroblasts with a test compound;
b) determining a level of activity of a collagen inhibiting gene of the fibroblasts contacted with the test compound;
c) comparing the level of activity of the collagen inhibiting gene to a control; and
d) identifying the test compound as a fibrotic agent capable of modulating a fibrotic process when the activity of the collagen inhibiting gene indicates an upregulation or downregulation of the collagen inhibiting gene relative to the control.

2. The method of claim 1, wherein a gene product of the collagen inhibiting gene is druggable.

3. The method of claim 1, wherein the level of activity of two or more collagen inhibiting genes is determined.

4. The method of claim 3, wherein the two or more collagen inhibiting genes are from the same gene family.

5. The method of claim 1, wherein the test compound is ribonucleic acid (RNA) selected from the group consisting of small interfering RNA, micro RNA, and small activating RNA.

6. The method of claim 1, wherein the activity of the collagen inhibiting gene is determined by at least one of protein quantitation and transcriptomic analysis.

7. The method of claim 6, wherein the activity of the collagen inhibiting gene is determined by measuring reporter gene activity, and wherein the protein is a fluorescent protein coded by a reporter gene inserted downstream of an endogenous promoter for the collagen inhibiting gene in the fibroblasts.

8. The method of claim 7, wherein the fluorescent protein is a red fluorescent protein selected from the group consisting of mCherry, mStrawberry, mOrange, and dTomato.

9. The method of claim 6, wherein the activity of the collagen inhibiting gene is measured by transcriptomic analysis, the transcriptomic analysis comprising:
a) generating a transcriptional profile for the plurality of fibroblasts contacted with the test compound, wherein the transcriptional profile comprises data related to the transcription of a collagen inhibiting gene,
b) comparing the transcriptional profile of the fibroblasts to a control, and
c) identifying the test compound as a fibrotic agent capable of modulating a fibrotic process when the transcriptional profile for the fibroblasts, relative to the control, indicates an upregulation or downregulation of the collagen inhibiting gene.

10. The method of claim 9, wherein the transcriptomic analysis further comprises isolating RNA from the fibroblasts, creating labeled cRNA or cDNA from the isolated RNA, and hybridizing the labeled cRNA or cDNA to a microarray comprising a probe for the collagen inhibiting gene.

11. The method of claim 1, further comprising administering the fibrotic agent to a person in need of treatment.

12. The method of claim 11, wherein administering the fibrotic agent results in an increase in collagen production compared to a placebo.

13. The method of claim 1, further comprising mixing the fibrotic agent with a carrier to provide a treatment composition.

14. The method of claim 13, wherein the treatment composition includes an additional ingredient selected from the group consisting of skin care actives, anti-fibrotic actives, pro-fibrotic actives, and combinations thereof.

15. The method of claim 13, wherein the treatment composition is in a form suitable for at least one of topical application, ingestion, or injection by a person.

16. The method of claim 1, wherein the fibroblasts are immortalized fibroblasts or transformed fibroblasts.

17. The method of claim 16, wherein the immortalized fibroblasts exhibit about the same endogenous expression and inducible expression of COL1A1 relative to their parental cell line.

18. The method of claim 1, wherein at least 25 compounds can be tested simultaneously.

19. A method of identifying fibrotic agents capable of modulating a fibrotic process, comprising:
a) contacting a plurality of immortalized or transformed fibroblasts with a test compound and a protein coded by a collagen inhibiting gene, wherein the protein is capable of modulating expression of a collagen synthesizing gene;
b) determining a level of activity of the collagen synthesizing gene by measuring collagen amount;
c) comparing the measured collagen amount to a control; and
d) identifying the test compound as a fibrotic agent capable of modulating a fibrotic process when the measured collagen amount increases or decreases relative to the control.

20. The method of claim 19, further comprising administering the fibrotic agent to a person in need of treatment.
